(19) United States
(12) Patent Application Publication
(10) Pub. No.: US 2003/0096020 A1
(43) Pub. Date: May 22, 2003

(54) Novel herbal composition for the management of bronchial asthma and a process of manufacturing the same

(76) Inventors: Narasimha Baba Brindavanam, Ghaziabad (IN); Chandrakant Katiyar, Ghaziabad (IN); Yadlapalli Venkateswara Rao, Ghaziabad (IN)

Correspondence Address: Bernhard D. Saxe, FOLEY & LARDNER, Washington Harbour, 3000 K Street, N. W., Suite 500, Washington, DC, 20007-5109, US

(21) Appl. No.: 09/881,042

(22) Filed: Jun. 15, 2001

(30) Foreign Application Priority Data
Jun. 4, 2001 (IN) 636/DEL/2001

(51) Int. Cl. A61K 35/78

(52) U.S. Cl. 424/725; 424/757; 424/769; 424/734

(57) ABSTRACT

The invention provides a novel oral liquid herbal composition useful in the management of asthma in diabetic and calorie conscious persons, said composition comprising a therapeutically effective amount of plant extracts selected from Solanum xanthocarpum, Albizzia lebbeck, Tribulus terrestris, Glycyrrhiza glabra, Pistachia integerrima, Adathoda vasica and Woodfordia fruticosa, and having self-generated ethanol to the extent of 7 to 11% v/v and not more than 1 to 3% w/w of sugar content.

### 1. FIELD OF INVENTION

The invention provides a novel herbal composition virtually free of sugars and containing limited self-generated alcohol, for the management of bronchial asthma, in the mammals, especially humans. The invention also provides a method for the manufacture of this herbal composition in liquid oral dosage form. This process of the invention facilitates the production of fermented liquid oral composition, which enhances the biological activity of the formulation for the management of asthma in human beings. The formulation also provides specific benefits to the diabetic patients suffering from bronchial asthma.

### 2. FIELD OF INDUSTRIAL APPLICATION

Bronchial asthma is a common chronic disease (asthma in short) wherein, the patient suffers from paroxysmal spells of breathlessness precipitated by narrowing of wind pipe and its branches. Between 100-150 million people around the globe reportedly suffer from asthma and this number is rising. World wide annual death from this condition has reached over 1,80,000. India alone has estimated 15-20 million asthmatics and a prevalence rate between 10-15% in 5-11 year old children. The management of bronchial asthma can be grossly divided into two components viz. Crisis management and maintenance therapy. While the available methods of crisis management are not disputed for the effectiveness, the modalities for maintenance therapies have been far from satisfactory. On the other hand, traditional systems of medicine like Ayurveda - provide useful leads to improve the quality of maintenance care for asthmatic patients. This invention allows the inventors to customize a unique herbal composition judiciously, and to develop a process for manufacturing liquid orals used in the management of asthma.

### 3. BACKGROUND OF INVENTION

### 3.1. DESCRIPTION OF BRONCHIAL ASTHMA

Bronchial asthma is a common chronic disease (asthma in short) due to reversible airway narrow caused by promoted reaction of trachea to all kinds of stimulation. The causes of bronchial asthma are complicated. Most of them are triggered by factors in- and -out of body on the basis of inheritance, such as inhaled substances, infection, climate, medicine, diet, mental factors etc. Symptoms can occur spontaneously or can be triggered by respiratory infections, exercise, cold air, tobacco smoking or other pollutants. The muscle of the bronchial tree becomes tight and the lining of the air passages get swollen, reducing the airflow and produces a wheezing sound. Mucus production is increased. Typically, the individual breathes relatively normally, and will have periodic attacks of wheezing.

It is likely that asthma attacks triggered by different factors have different mechanisms that ultimately lead to the narrowing of air passages. Three leading theories are currently discussed to explain the mechanism of asthma (Leff A.R. Future Directions in Asthma Therapy. Is a cure possible? Chest 1997 Feb: 111(2) Suppl.: 61s-68s).

The first one is that asthma is a fundamentally allergic sequence due to a wrongful response of the immune (defense) to a challenge, e.g. by inhaled pollutants.

The second theory is the “neurogenic hypothesis” that asthma attacks are precipitated by a sudden spasm of smooth muscles in the air passage due to imbalance within the nervous system i.e autonomous nervous system which regulates smooth muscles via β-receptors and a-receptors

The third theory “myogenic hypothesis” explains that white cells migrating to walls of air passages make the smooth muscles hyperactive and prone to sudden spasms leading to an attack of asthma.

According to the WHO fact sheet, 100-150 million people around globe suffer from asthma and the number is rising. World-wide, death from this condition has reached over 1,80,000 annually. In the United States alone, the number of asthmatics has increased by over 60% since 1980 and deaths have doubled to 5000 a year. Asthma is not just a public health problem for developed countries. In the developing countries, the incidence of the disease varies greatly. India has an estimated 15-20 million asthmatics. In Brazil, Peru and Uruguay, prevalence of asthma symptoms in children varies from 20-30%. In India, rough estimates indicate prevalence of between 10-15% in 5-11 year old children. The human and economic burden associated with this condition is severe. The cost of asthma to society can be reduced to a large extent through concerted international and national efforts. It is estimated that the world-wide, the economic costs associated with asthma are estimated to exceed those of TB and HIV/AIDS. In US for example, the annual asthma care cost (direct and indirect) exceeds US$6 billion.

Asthma cannot be cured, but can be controlled. The strongest risk factors for developing asthma are exposure, especially in infancy to indoor allergens and a family history of asthma and allergy. Because asthma is a chronic recurrent condition, it usually requires continuous medical care. Patients with moderate to severe asthma have to take long-term medication daily to control the underlying inflammation and to prevent the asthmatic spell. When an asthmatic attack occurs, the crisis management regimen should be switched to.

Medication is the only way to control asthma. It is also important to avoid asthma triggers - stimuli that irritates and inflame the airways. WHO recognizes asthma as a disease of major public health importance and plays an important role in the co-ordination of international efforts against the disease. These include increase in public awareness of the disease, organizing and co-ordinating global epidemiological surveillance to monitor global and regional trends in asthma and to develop appropriate strategies for its management and prevention.

Currently, the treatment of asthma includes the following group of drugs.

1. Adrenoceptor stimulants (selective β2stimulants, selective β2-agonists)

2. Glucocorticoids which inhibit inflammatory changes in airways

3. Bronchodialators which help open airways and also prevent bronchitis

4. Cromones similar in action as glucocorticoids, but weaker

5. Anticholinergics which decrease airways mucus secretion β2-receptor agonists stimulate cyclic AMP and inhibit release of substances from inflammatory cells leading to airway narrowing i.e leucotrienes, prostaglandins, histamines, acetylcholine etc. Steroids (Glucocorticoids) are effective inhibitors of inflammatory messengers which directly or indirectly produce narrowing of air passage. They are also known to suppress immune system and thereby reduce the risk of allergy. Bronchodialators comprise the third line of treatment for patients with asthma and mostly for crisis management. This is used for the relief bronchospasm. They may also block the late inflammatory events after exposure to allergens. Cromones are involved in the mast cell stabilisation and therefore, are shown effective in controlling asthma induced by allergens, exercise, drugs etc. Leucotrienes which are released by inflammatory cells are pivotal in the development of asthma. Anticholinergic drugs are designed to block the expression of leucotrienes in airways.

With the advances in drug research, the crisis management for bronchial asthma is very much simplified. However, the drug modalities employed in maintenance therapy are far below satisfaction. For example, regular use of bronchodialators has a deleterious effect on the functioning of the heart. Over and above, they are known to cause drug resistance rapidly. Frequent use of glucocorticoids cause generalized fluid retention and also weaken the bone mineral mass. Further, they are known to weaken the immune system causing a risk of frequent infections. On the other hand, natural products are being evaluated to evolve multiple responses in order to control the risk of frequent attacks of breathlessness. Many herbal drugs are reported to cause selective immune suppression and cause mast cell stability.

### 3.2. Herbal Drugs and Bronchial Asthma

In Ayurveda several herbal ingredients are mentioned for the treatment of bronchial asthma (Tamaka Shwasa). Herbal ingredients such Kantakari (Solanum xanthocarpum), Shireesh chhal (Albizzia lebbeck), Yashthi madhu (Glycyrrhiza glabra), etc. are few examples in this category. Various scientific investigations on these plants suggest their role in the care of asthma. Some of the herbal formulations meant for the maintenance therapy of bronchial asthma available in the market are perhaps based on these leads.

Numerous reports are available on the role of herbal ingredients in the management of bronchial asthma. Govindan S et al has reported the clinical efficacy of Solanum xanthocarpum in bronchial asthma (J Ethnopharmacol 1999 Aug; 66(2):205-10). Oral administration of the whole plant in the form of powder significantly improved various parameters of pulmonary function in asthmatic patients. Another study indicated the role of Kantakari in chronic bronchitis, bronchial asthma and non-specific productive cough (J Assoc Physicians India 1971 Oct; 19 (10): 741-4).

Albizzia lebbeck commonly known as Shireesh, is used as an anti-convulsant and in the treatment of allergy. The role of Albizzia lebbeck in bronchial asthma and eczema was evaluated. Tripathi RM et al has reported the role of Albizzia lebbeck on the deregulation rate of sensitised peritonial mast cells of albino rats when challenged with antigen (Tripathi RM, Sen PC and Das PK (3 Ethnopharmacol 1979 Dec; 1(4): 385-96). The in vitro effects of Albizzia lebbeck and DCG on the deregulation rate of the sensitized mast cells were also studied. The results suggest that, it have a significant cromoglycate like action on the mast cells.

Adathoda vasica is a well known plant drug in Ayurveda and Unani medicine. It has been used for the treatment of various diseases and disorders, particularly for respiratory tract infections. Paliwa JK et al reported the anti-allergic activity of the active component isolated from the leaves and roots of Adhatoda vasica (Paliwa JK, Dwivedi AK, Singh S and Gupta RC, Int J Pharm 2000 Mar 20; 197(1-2):213-20). Dhuley JN has reported the antitussive avtivity of Adhatoda vasica extract on mechanical or chemical induced stimulation-induced coughing (Dhuley JN, J Ethnopharmacol 1999, Nov 30; 67 (3):361-5). Dorsch W et al has reported the role of alkaloids isolated from Adhatoda vasica (Dorsch W, Wagner H; Int Arch Allergy Appl Immunol 1991; 94 (1-4):262-5). The study revealed that the alkaloids have shown protection against allergen-induced bronchial obstruction in guinea pigs. Glycyrrhiza glabra is widely being used in the formulations meant for bronchitis (James A. Duke, CRC Handbook of Medicinal Plants, CRC press, Inc. Florida, pg- 215). Nafyrov KM et al has reported the anti-histaminic activity of Glycyrrhizinic acid and its derivatives (Nafyrov KM et al, Farmakol Toksikol, 43(4), pg 399, 1980).

3.3. Liquid Orals in Ayurveda:

Asavas and Arishtas comprise an important group of liquid orals mentioned in ancient Ayurvedic literature. This dosage form offers the advantages of accessibility, palatability and product stability over the traditionally used decoctions, which are primitive in nature. This particular group of liquid orals finds use not only in Ayurveda, but also in other traditional systems of medicine practiced in neighboring oriental countries like Sri Lanka, Tibet, China and Bhutan. A fairly large number of Ayurvedic formulations described in ancient and mediaeval Ayurvedic literature fall under this group. Various Ayurvedic pharmaceutical companies in India manufacture almost 50 different formulations falling under this range and some of these formulations are also being exported to other countries. To an estimate, every year Rs. 2000 million worth of Asava & Arishta group of formulations are being consumed in India alone.

3.4. Process of Asavas/Arishtas: An outline

Asava-Arishta group of formulations is being manufactured as per the procedures laid down in ancient Ayurvedic texts. This procedure involves mainly three phases: 1) Extraction 2) Preparation of fermentation medium 3) Inoculation and fermentation and these three phases are summarized as below:

Extraction Phase: Water is used as solvent for extraction purposes. For Asavas, the extraction procedure involves a cold infusion technique. Therefore, the coarse powder of herbal material is soaked in water for a fixed duration at room temperature and a filtrate is obtained. In case of Arishtas, the extraction procedure involves an Open Pan Boiling Technique. Accordingly, the coarsely ground herbal material is charged to a boiling pan along with required quantity of water. This mixture is then, heated using steam or firewood or some other fuel. The filtrate is then collected discarding the herbal marc.

Preparation of fermentation medium: To grow any microorganism nutrients are necessary. As the extract mainly contains active constituents coming from herbs, Jaggery was been suggested as a source of complex nutrition in ancient literature. Accordingly, the fermentation medium is to be prepared by dissolving Jaggery in the herbal extract obtained either by cold infusion for Asavas or by means of Open Pan Boiling Technique for Arishtas.

Inoculation & Fermentation: Ancient literature recommends two herbs-viz. Woodfordia fruticosa and Madhuca latifolia as the inoculum bearing herbs. One or both of these two inoculum-bearing herbs and other aromatic herbs in form of fine powders are topped to the fermentation medium. Fermentation is carried out in a closed fermentation vessel, under controlled temperature conditions, at 30-35 °C. for a period of 40-45 days. The end product is expected to contain self-generated alcohol in range of 7 to 11% v/v.

The herbal product range produced as per the above process are termed as Asava or Arishta as the case may be. The product line has certain specific advantages from clinical application and stability points of view and the same can be summarized as under:

Products prepared under the process are generally palatable for the user with a sweet taste combined with a fine spicy aroma, which masks the unacceptable taste and odour of the active, herbal ingredients.

Apart from the aqueous extract of the prime ingredients, the preparation brings the hydro-alcoholic extract of supportive ingredients used as powders added during fermentation.

The process also brings the extract of inoculum bearing herbs, which is considered to potentiate the activity of prime ingredients.

The self-generated alcohol content in the product acts as a preservative and contributes to its prolonged shelf life.

Self-generated alcohol is considered to enhance the bioavailability of all the herbal ingredients contained in the formulation.

As seen from the above information Asavas and Arishtas have many advantages and they constitute a brilliant pharmaceutical concept. Classical Ayurvedic texts books described formulations falling under this category for management of bronchial asthma. For example Kanakasava (Bhaishajya Ratnavali) falls in this category. However, this kind of available formulations are generally administered only to non-diabetic asthmatic patients. Considering various factors the concomittent occurrence of diabetics and bronchia asthma in the same patient is on the rise. For these patients, the existing formulations cannot be employed and risking an increase in the blood sugar level.

Generally all the Asavas and Arishtas contain 7-11 %v/v of alcohol content and about 20%w/w of sugar content. It is well known fact that, in the presence of alcohol the assimilation of carbohydrate such as sugars is very high. This practically means that if a diabetic patient takes any Asavas and Arishta formulation, the entire sugar content gets assimilated into the body and there is a great risk of immediate rise in the blood glucose levels. Considering this risk, generally Ayurvedic physicians do not recommend this type of formulation to any diabetic patient. Patient having both diabetes and bronchial asthma cannot be offered oral formulations from the category ‘Asavas’ and ‘Arishtas’.

Looking at the process intricacies of Asava/Arishta, inventors noted that if a preparation is made under this category for the management of bronchial asthma with an added advantage to the diabetic patients, it could provide therapeutic benefits to a larger segment of the population.

Thus, the present invention, relates to a herbal composition useful in the management of bronchial asthma in both, diabetic and non-diabetic patients. This composition is virtually free from residual sugars and therefore, suitable for asthmatic diabetic patients and calorie conscious consumers.

The invention also provides a method by which a specific herbal composition for asthma can be prepared without much of residual sugar and be useful for the management of Bronchial asthma, both in normal and diabetic patients.

4. Objectives of the Invention:

The main object of the invention is to provide a herbal composition falling under the category “Asavas” and “Arishtas”, having final sugar content of upto 3% w/w and useful in the management of bronchial asthma especially in the case of diabetics and calorie conscious non-diabetics.

Another object of the invention is to provide a process for the manufacture of the said herbal composition, virtually free of sugar.

Still another object of the invention, is to prepare this herbal composition using the process pathways of classical Asavas and Arishtas

Yet another object of the invention is to develop a novel process of manufacturing anti- asthmatic Asavas and Arishtas containing less than 1% w/w of residual sugars and containing limited amount of self generated alcohol.

### 5. SUMMARY OF THE INVENTION

The invention provides a novel herbal composition useful in the management of bronchial asthma, especially in case of diabetics and calorie conscious persons, said composition having final sugar content of upto 3% w/w.

The invention also provides a process for the manufacture of the said such composition.

### 6. DETAILED DESCRIPTION OF THE INVENTION

Thus, the invention provides a novel oral liquid herbal composition useful in the management of asthma, said composition comprising a therapeutically effective amount of plant extracts, self-generated ethanol to the extent of 7 to 11% v/v and having not more than 1 to 3% w/w of sugar content.

Specifically, this oral liquid herbal composition comprises extracts of plants selected from:

| | | |
|---|---|---|
| a. | Solanum xanthocarpum | 3-12% w/v |
| b. | Albizzia lebbeck | 5-15% w/v |
| c. | Tribulus terrestris | 3-12% w/v |
| d. | Glycyrrhiza glabra | 1-10% w/v |
| e. | Pistachia integerrima | 1-5% w/v |
| f. | Adathoda vasica | 1-5% w/v |
| g. | Woodfordia fruticosa | 1-7% w/v |

and has sugar content to the extent of 1 to 3% w/w, self-generated ethanol to the extent of 7 to 11%, and, optionally, comprises extracts/power of Elettaria cardamom 0.1 to 00.3% w/v, Piper longum (0.1 to 0.3%), Mesua ferrea 0.1 to 0.3% w/v and Syzigium aromaticu 0.1 to 0.3% w/v.

In an embodiment, the oral liquid herbal composition comprises:

| | | |
|---|---|---|
| a. | Solanum xanthocarpum | 7% w/v |
| b. | Albizzia lebbeck | 9% w/v |
| c. | Tribulus terrestris | 7% w/v |
| d. | Glycyrrhiza glabra | 5% w/v |
| e. | Pistachia integerrima | 2% w/v |
| f. | Adathoda vasica | 2% w/v |
| g. | Woodfordia fruticosa | 3% w/v |

and has sugar content to the extent of 1 to 3% w/w, self-generated ethanol to the extent of 7 to 11% v/v, and, optionally, comprises extracts/power of Piper iongum (0.075% w/v), Elettaria cardamomum (0.125% w/v), Syzigium aromaticum (0.075% w/v), and Mesua ferrea (0.100% w/v).

The plant parts used for preparation of the said composition are fruits, leaves, stem and flowers of the plants.

This oral liquid herbal composition of the invention is manufactured by the process comprising the steps of:

a. obtaining the extract of plant parts,

b. adding nutrients to the extract of step (a) in a manner such that the sugar content in the culture medium does not exceed 20% w/w,

c. adding micro-organisms capable of fermentation to the culture medium of step (b) and allowing it to ferment until the self-generated ethanol content thereof reaches 7 to 11% v/v and

d. obtaining a herbal composition having total sugar content of not more than 3%

It is the Applicant's finding that

addition of herbs like Piper longum, Eletteria cardamomum, Syzigium aromaticum and Mesua ferrea to the composition enhances the activity of the composition. These ingredients act as ‘drug potentiators’. Their role is to invoke positive responses in the body so that the drug can work effectively. They also play an important role as bio-availability enhancers in the body.

the herbal composition of the invention is most effective only in liquid form and not in any other physical form. In fact, the Applicant even tried to formulate the composition in other physical forms such as tablets or capsules. These forms were found highly ineffective.

The Applicants have also found that although, the prior art gives the lists of few herbs such as Solanum xanthocarpu, Albizzia lebbeck, Adathoda vasica and Glycyrrhiza glabra for treatment of bronchitis, Tribulus terrestris and Pistacia integerrima also play an important role in alleviating bronchitis. The extracts of these herbs helps in warding off allergens and also plays a positive role in drug metabolism in the body. Therefore, the applicant has combined these herbs along with Solanum xanthocarpu, Albizzia lebbeck, Adathoda vasica and Glycyrrhiza glabra to achieve a novel herbal composition.

As for preparation of the composition, the plant parts are obtained by cold infusion or hot decoction methods. The cold infusion method comprises the steps of extracting the plant parts in water at a temperature ranging between 20° C. to 30° C. The hot decoction method comprises the step of extracting the plant parts in water by heating at a temperature in the range of 60° C. to 90° C.

The nutrient used in the process is a complex nutrient like jaggery or simple sugar like glucose, fructose or any other hexose sugar. The nutrient is in physical forms such as solid or liquid. The nutrients are added by fed batch or batch fermentation method.

The nutrients are added in small amounts in regular intervals (at gradient quantities) in fed batch fermentation method. The addition of nutrients in fed batch fermentation method is such that each batch of nutrients added (each gradient) does not impart more than 5%v/v of sugar and the overall quantum of sugar added in the entire process does not exceed 20% w/w. The nutrients are added to the medium in the beginning at once in batch fermentation method.

The microorganisms for fermentation comprise micro-organisms obtained from conventional sources like Woodfordia fruticosa, pure cultures such as baker's yeast, alcohol producing strains of Saccharomyces sp or strains of Saccharomyces cereviceae such as DRF-UDS-004/Wf, DRF-UDS-016/Wf, DRf-UDS-017/Wf or a combination thereof. These strains of Saccharomyces cereviceae i.e. DRF-UDS-004/Wf, DRF-UDS-016/wf and DRF-UDS-017/Wf have been deposited at the Microbial Type Culture Collection (MTCC), Chandigarh, India and are available and accessible to the public. These strains are also deposited at _______ depository and bear Accession numbers ______ respectively.

The culture medium used in the process is incubated at a temperature ranging between 20° C. to 37° C. for 2 to 40 days under anaerobic conditions maintaining the pH of the medium from 4 to 6. The temperature is preferably maintained at 30° C. The incubation is effected for 2 to 8 days preferably for 4 days under aerobic conditions. The pH of the culture medium ranges from 4 to 6 and is maintained at 4.5. The resultant composition of the above process is one wherein the final sugar content is not more than 1 to 3% w/w and generally maintained at less than 1 %w/w.

The invention pertains to the management of bronchial asthma wherein herbal formulations have an edge of safety and efficacy over the conventionally used synthetic drug molecules

In an embodiment, the invention delivers the specific advantages of Asavas and Arishtas over the conventionally used solid dosage forms. For this purpose the invention seeks to find solution to important limiting factor of the range - viz. the residual sugar content.

In another embodiment, the invention goes to the very fundamental aspects of fermentation procedure and identifies a simple solution to overcome the problem residual sugars in ‘Asava Arista’formulations.

The present invention deals with a method of manufacturing sugar free fermented liquids under the category of Asavas and Arishtas.

The inventive steps for this invention can be divided under the following major headings:

a) Identification, development of a herbal composition comprising of known herbal drugs for asthma. Each 100 ml of the finished product consists of the extracts obtained from:

| | |
|---|---|
| Solanum xanthocarpum (whole plant) | 3-12 Gm |
| Albizia lebbeck (Bark) | 5-15 Gm |
| Tribulus terrestris (fruits) | 3-12 Gm |
| Glycyrrhiza glabra (Root) | 1-10 Gm |
| Pistacia integerrima (Galls) | 1-5 Gm |
| Adathoda vasica (Leaves) | 1-5 Gm |
| Woodfordia fruticosa (Flower) | 1-7 gm |

b) The second phase of inventive steps included, identification of certain drug potentiators and identify a process pathway to incorporate the same into a liquid oral dosage form. The following drug potentiators are identified to enhance the anti-asthma activity.

| | |
|---|---|
| Elettaria cardamomum (Fruits) | 0.1-0.3 Gm |
| Piper longum (Fruits) | 0.1-0.3 Gm |
| Syzigium aromaticum (Flower buds) | 0.1-0.3 Gm |
| Mesua ferrea (Anthers) | 0.1-0.3 Gm |

The Applicants have conducted a series of experiments mainly with reference to the above inventive steps. These experiments were essentially designed on a simple biological principle that a complex sugar is inverted into simple sugars by the microorganism to produce alcohol in any medium. When a complex sugar is added in the medium as per traditional formula, only a part of them are inverted and rest of sugars remain as residual sugars in the finished product.

On the other hand, judicious design of the fermentation medium with simple sugars might help to overcome this problem and render the finished product virtually sugar free. Several experiments were conducted using different kinds of sugars such as Jaggery, Sucrose, Glucose, Invert Syrup etc. These studies have invariably proved that the residual sugars can be controlled by means of judicious use of simple sugars in the fermentation medium.

The present invention provides novel product having a therapeutic benefit in the maintenance regimen of bronchia asthma without any side effects over prolonged usage and comprising the following ingredients.

| Botanical Name | Common Name |
|---|---|
| Solanum xanthocarpum | Kantakari |
| Albizzia lebbeck | Shireesh |
| Tribulus terrestris | Gokshur |
| Glycyrrhiza glabra | Yashthimadhu |
| Pistacia integerrima | Karkatashringi |
| Adathoda vasica | Vasaka |
| Woodfordia fruticosa | Dhatki pushpa |
| Piper longum | Pippali |
| Elettaria cardamomum | Elaichi |
| Syzigium aromaticum | Lavang |
| Mesua ferrea | Nagkeshar |

The invention is illustrated by the following examples which should not be construed as limitations on the inventive concept embodied herein.

### EXAMPLE 1

Herbal ingredients such as Kantakari (Solanum xanthocarpum), Shireesh chhal (Albizzia lebbeck), Gokshur (Tribulus terrestris), Yashthi madhu (Glycyrrhiza glabra), Karkatashringi (Pistacia integerrima), Vasaka (Adathoda vasica) are coarsely ground to small pieces and extracted twice with water using boiling pan. The extract thus obtained was dispensed into 2 sets of Erelynemayor flasks. In one set of flasks jaggery was dissolved as nutrient for fermentation. In another set of flasks invert syrup was added. To both sets of flasks Woodfordia fruticosa and spicy materials such as Piper longum, Elettaria cardamomum, Syzigium aromaticu Mesua ferrea were topped and these flasks were incubated at 30° C without shaking. The samples were estimated at regular intervals for alcohol generation and residual sugar content.

The results of final round of analysis of this experiment are tabulated below:

| Experiment | Final Sugar Content | Alcohol Content |
|---|---|---|
| Conventional nutrient | 24% w/w | 10.2% v/v |
| Modified nutrient | 0.8% w/w | 9.1% v/v |

The amount of alcohol produced at the end of fermentation is 7-11% v/v. Results indicated that, samples prepared using a conventional nutrient has shown 20 %w/w of residual sugars. However, samples prepared with invert syrup contain less than 1 %w/w of residual sugars. The above example clearly indicates that, by an ingenious modification of nutrient in the culture medium, it is possible to control the residual sugar content in the finished product.

### EXAMPLE 2

Herbal ingredients such Kantakari (Solanum xanthocarpum), Shireesh chhal (Albizzia lebbeck), Gokshur (Tribulus terrestris), Yashthi madhu (Glycyrrhiza glabra), Karkatashringi (Pistacia integerrima), Vasaka (Adathoda vasica) are coarsely ground to small pieces and extracted twice with water using boiling pan. The extract thus obtained was transferred to a Fermentor, Bioflo 3000. To this extract invert syrup was added. To this medium, Woodfordia fruticosa and powders of Piper longum, Elettaria cardamomum, Syzigium aromaticum, Mesua ferrea were topped over the fermentation medium. The temperature of the fermentation medium was maintained at 3⁰° C. The samples were estimated at regular intervals for alcohol generation and residual sugar content.

The results of this experiment are tabulated below:

| Experiment | Final Sugar Content | Alcohol Content |
|---|---|---|
| Modified Method | 0.7% w/w | 9.3% v/v |

The amount of alcohol produced at the end of fermentation is 7-11% v/v and the residual sugars content in the fermentation medium was less than 1%w/w. It is further confirmed that the end results of the process covered by example-i remain the same irrespective of the batch size.

### EXAMPLES 3

Herbal ingredients such Kantakari (Solanum xanthocarpum), Shireesh chhal (Albizzia lebbeck), Gokshur (Tribulus terrestris), Yashthi madhu (Glycyrrhiza glabra), Karkatashringi (Pistacia integerrima), Vasaka (Adathoda vasica) and Woodfordia fruticosa are coarsely ground to small pieces and extracted twice with water using boiling pan. The extract thus obtained was dispensed into 5 sets of Erelynemayor flasks. Invert syrup was added to all flasks as a source of nutrient. To this spicy materials such as Piper longum, Elettaria cardamomum, Syzigium aromaticum, Mesua ferrea were topped and these flasks were inoculated with different microorganisms. The first set of flasks were inoculated with Baker's yeast whereas the rest of 4 sets were inoculated with yeast cultures labeled as, DRF-UDS -004IWF, DRF-UDS-16/WF, DRF-UDS-17/WF individually and a combination of all three strains. All the flasks were incubated under controlled conditions allowing a complex anaerobic fermentation. The samples were estimated at regular intervals for alcohol generation and residual sugar content.

The results of this experiment are tabulated below:

| Experiment | Final Sugar Content | Alcohol Content |
|---|---|---|
| Baker's yeast | 0.6% w/w | 9.2% v/v |
| DRF-UDS-004/WF | 0.9% w/w | 9.5% v/v |
| DRF-UDS-16/WF | 0.7% w/w | 9.1% v/v |
| DRF-UDS-17/WF | 0.8% w/w | 9.0% v/v |
| Mixed Culture of DRF- | 0.6% w/w | 9.7% v/v |
| UDS | | |

Experimental results suggested that the process is effective irrespective of type of fermenting microorganism. The micro-organisms DRF-UDS-004/Wf, DRF-UDS-016/Wf and DRF-UDS-017/Wf are three different strains of Saccharomyces cereviceae and are available to the public as they are deposited at Microbial Type Culture Collection (MTCC), Chandigarh, India. Each of these strains exhibit different characteristics as under:

The characteristics of the yeast strain DRF-UDS 004/wf are as under:

a) being sugar resistant,

b) capable of producing alcohol to the extent of about 7 to lIv/v% continuously and consistently in a herbal extraction medium,

c) capable of overcoming resistance conferred by the herbal and spicy ingredients present in the medium,

d) capable of attaining biomass of about 1 to 4g/l and potentiating and enhancing the therapeutic value of the herbal formulation, and

e) exhibiting the following bio-chemical properties:

i) urease test - positive,

ii) utilization of fructose test - negative,

iii) starch hydrolysis test - negative,

iv) xylose hydrolysis test - negative.

The characteristics of the yeast strain DRF-UDS 0 1 6Iwf are as under:

a) being sugar resistant,

b) capable of producing alcohol to the extent of about 7 to 1 lv/v% continuously and consistently in a herbal extraction medium,

c) overcoming resistance conferred by the herbal and spicy ingredients in the medium,

d) capable of attaining biomass of about Ito 4g/l and potentiating and enhancing the therapeutic value of the herbal formulation, and

e) exhibiting the following bio-chemical properties:

i) urease test - negative,

ii) utilization of fructose test - positive,

iii) starch hydrolysis test - positive,

iv) xylose hydrolysis test - positive.

The characteristics of the yeast strain DRF-UDS 0 1 7/wf are as under:

a) being sugar resistant,

b) capable of producing alcohol to the extent of about 7 to 1 lv/v% continuously and consistently in a herbal extraction medium,

c) overcoming resistance conferred by the herbal and spicy ingredients in the medium,

d) capable of attaining biomass of about 1 to 3.5g/l and potentiating and enhancing the therapeutic value of the herbal formulation, and

e) exhibiting the following bio-chemical properties:

i) urease test - negative,

ii) utilization of fructose test - positive,

iii) starch hydrolysis test - positive,

iv) xylose hydrolysis test - negative.

### EXAMPLE 4

Herbal ingredients such Kantakari (Solanum xanthocarpum), Shireesh chhal (Albizzia lebbeck), Gokshur (Tribulus terrestris), Yashthi madhu (Glycerrhiza glabra), Karkatashringi (Pistacia integerrima), Vasaka (Adathoda vasica) and Woodfordia fruticosa are coarsely ground to small pieces and extracted twice with water using boiling pan. The extract thus obtained was transferred to a Fermentor, Bioflo 3000. The nutrient (Invert Syrup) was added in a fed batch mechanism. The medium was inoculated with mixed culture of DRF-UDS-004/wf, DRF-UDS-016/Wf and DRF-UDS-017/Wf and spicy materials such as Piper longum, Elettaria cardamomum, Syzigium aromaticum, Mesua ferrea were topped over the fermentation medium. The temperature of the fermentation medium was maintained at 30° C. Samples were estimated at regular intervals for alcohol generation and residual sugar content. The amount of alcohol produced at the end of fermentation was 7-11% v/v and the residual sugars content was less than 1%w/w. This particular fed batch mechanism shall have the advantage of avoiding any kind of foaming problem during fermentation. These experimental results further emphasize the effectiveness of the process and the end results of the process are same irrespective of the type of fermentation.

### EXAMPLE 5

To examine the comparative efficacy of solid and liquid oral dosage forms the following preparations were made.

Sample A: Liquid oral sample was prepared using a conventional nutrient and process. Such sample contains 20% w/w of the residual sugar.

Sample B: Sample was prepared by the novel process mentioned in the description which contains less than 1 %w/w of residual sugar content.

Sample C: Placebo liquid formulation (without any herbal ingredients, but fermented liquid).

Sample-D:The following individual herbs are coarsely ground to small pieces and extracted twice with water using boiling pan. The filtrate is then collected and was dried to obtain a fine powder (Quantities mentioned here are equivalent to 100 ml of liquid oral preparation)

| | |
|---|---|
| Solanum xanthocarpum (whole plant) | 3-12 Gm |
| Albizia lebbeck (Bark) | 5-15 Gm |
| Tribulus terrestris (Seeds) | 3-12 Gm |
| Glycyrrhiza glabra (Rhizome) | 1-10 Gm |
| Pistacia integerrima (fruit) | 1-5 Gm |
| Adathoda vasica (Leaves) | 1-5 Gm |
| Woodfordia fruticosa (Flower) | 1-7 gm |

To the above extract dry powders blend of following herbs was added.

| | |
|---|---|
| Piper longum | 0.1-0.3 Gm |
| Elettaria cardamomum | 0.1-0.3 Gm |
| Syzigium aromaticum | 0.1-0.3 Gm |
| Mesua ferrea | 0.1-0.3 Gm |

EXPERIMENTAL EVALUATION

1. Active anaphylaxis method to evaluate the mast cell count

2. To evaluate spasmolytic activity in isolated guinea pig ileum

3. To assess antihistaminic activity in guinea pig.

EXPERIMENTAL STUDIES

Active anaphylaxis method to evaluate mast cell count. The study was conducted on sparague-Dawley male healthy rats weighing 180-200 gm. All the rats were fed adlib on Hind-i lever feed with adequate tap water. They were housed in poly-propylen cages at room temperature.

COLLECTION OF SERUM SAMPLES

Sheep blood was freshly collected in glass tubes from the slaughter house and was allowed to clot at room temperature for 24 hrs. Straw coloured serum from the top was carefully poured into centrifuge tubes without disturbing the clot. The serum was centrifuged for 15 minutes at 3OOrpm and clear serum was carefully pipetted out into clean vials. These serum vials were stored at 4° C. They were used for sensitising the animals for active anaphylaxis, to challenge the mesenteric pieces and to challenge rats subjected to passive anaphylaxis.

ACTIVE ANAPHYLAXIS

Sensitization procedure: 0.5 ml of Triple antigen containing Diphtheria, Tetanus toxoid and Pertusis organisms was injected along with sheep serum at two different sites of lower abdomen.

Procedure: On the 5^{th} day of sensitization, rats were divided into 5 groups viz. Gr I (Control), Gr II (Standard), Group III (Sample A, 10 ml/kg/day), Group IV (Sample B, 10ml/kg/day), Group V (Sample C, 10ml/kg/day) and Group VI (Sample D, 50 mg.kg/day). Each group comprised of 12 animals. Group I and Group II received water and Prednisolone respectively (10 mg/kg/day - orally) up to 13th day. Group III, IV, V and VI received sample A, sample B, sample C (10 ml/kg/day) and sample D (200 mg/kg/day) respectively. On the 13^{th} day, 6 animals from each group were sacrificed for mast cell count. The results are shown in the Table 1. Rest of the animals were sacrificed on 21^{st} day. The animals did not receive any treatment between day 14 to day 21.

TABLE 1
The effect of sample A, B and C on 13^{th} and 21^{st} day of sacrifice in Active
anaphylaxis method

| | Degranulation (% mean +/− SEM) of mast cells | | | |
|---|---|---|---|---|
| Name of the | 13^{th} day sacrifice | | 21^{st} day sacrifice | |
| sample | central zone | peripheral zone | central zone | peripheral zone |
| Control | 54.51 ± 30 | 32 ± 2.72 | 56.32 ± 11.28 | 59.06 ± 5.6 |
| Standard | 8.4 ± 2.0*** | 8.7 ± 1.621*** | 29.4 ± 7.2*** | 37.51 ± 8.3** |
| Sample A | 7.35 ± 1.91*** | 10.23 ± 2.05** | 31.36 ± 2.01** | 33.22 ± 2.03* |
| Sample B | 7.30 ± 1.81*** | 10.26 ± 1.98** | 31.16 ± 1.98** | 33.32 ± 2.03* |
| Sample C | 43.6 ± .28 | 12.42 ± 3.05 | 46.17 ± 6.85 | 58.54 ± 7.48 |
| Sample D | 29.64 ± 9.16 | 11.82 ± 4.92 | 39.98 ± 7.23 | 46.74 ± 8.16 |

Note:
**p < 0.01 (significant)
***p > 0.001 (Highly significant)

As seen from the above results, Sample A and Sample B have a statistically significant “antihistaminic” effects. Though sample D exhibited some degree of such effects, it is satistically not significant. This example illustrates that, the liquid oral show a higher degree of efficacy as compared to a solid dosage form. Therefore, further studies on herbal composition were done only with liquid oral dosage forms.

Evaluate the Spasmolytic Activity in Isolated Guinea Pig Ileum (Dale-Schultz Reaction)

Guinea pigs of either sex weighing 400-600 grn were used. Guinea pigs were sacrificed by stunning method and quickly dissected out. A portion of ileum, 2cm away from caecum was taken for isolated experiments. Tissue bath containing physiological salt solution maintained at 37° C.±0.5 with a pH of 7.4. Tissue was aerated with physiological salt solution (P.S.S). A mixture of Oxygen and Carbon dioxide was bubbled through PSS. The tissue was contacted through a silk suture to isometric force displacement, transducers which was coupled with physiograph (2001μ/v) to record the tissue activity. After 15 minutes of equilibrium, a sub maximal dose of histamine (0.2 μg/ml) for 1 minute and washed. After 15 minutes of equilibrium, tissue was tested for test drugs (1 μg/ml) i.e. sample A, Sample B and Sample C with histamine. The tissue was allowed to rest for at least 15 minutes between two doses. Finally the test samples at high concentration (10 μg/ml) were individually tested for their activities.

TABLE 2
The height and % age inhibition of histamine induced contractions

| | | | | Height | |
|---|---|---|---|---|---|
| | | | % age | of con- | |
| | | | res- | trac- | |
| S.No. | Drug | Dose | ponse | tion | % age |
| 1 | Histamine | 100 μg | 100 | 39 mm | 0 |
| 2 | Sample A | 1 ml | 7.69 | 3 mm | — |
| 3 | Sample A + Histamine | 1 ml + 10 μg | 15.38 | 6 mm | — |
| 4 | Sample B | 1 ml | 7.89 | 3.5 mm | — |
| 5 | Sample B + Histamine | 1 ml + 10 μg | 16.2 | 7 mm | — |
| 6 | Sample C | 1 ml | 56.41 | 22 mm | 43.59 |
| 7 | Sample C + Histamine | 1 ml + 10 μg | 82.05 | 32 mm | 17.95 |

The results shown in the above table signifies that the minimal contractions are observed with sample A and Sample B there by suggesting an equipotent anti spasmodic effect.

Evaluation of Antihistaminic activity of Sample A, Sample B and Sample C with Histamine Challenge in Guinea Pigs

Method

Guinea pigs (Both sexes) weighing between 200-250 gm were used.

1. A preconvulsant time was determined by exposing the animals to 1% histamine aerosol where the animals were observed for difficulty in breathing 2 days prior to administration of test drugs.

2. Animals were divided in to 10 groups (n-6 in each group) one group received water orally (control). Three groups received Sample A (group one receiving the drug at the dose of ¼ ml per 100 gm, second group-½ ml per 100 gm and third group-i ml per 100 gm of body weight). Among the remaining six groups three groups received sample B and the another three groups received Sample C at the similar dosage levels as that of Sample A.

3. The drugs were administered half an hour prior to exposure to histamine aerosol and then exposed to the aerosol at ½ hr, 1 hr and 2 hrs post treatment.

4. If the exposed animals did not show any signs of bronchospasm at the end of 120 mins. They were considered as to be protected.

When animals were exposed to 1% histamine aerosol average preconvulsant time was 35+/−6 sec. Average convulsant and death time was 62±7 sec. Sample A and Sample B at the doses of 1/4, 1/2and 1 ml per 100gm of body weight provided protection when challenged with histamine aerosol after 30 min, 1 hr and 2 hr of the drug administration. Maximum protection at 1 hr time point.

The results of this experiment are summarized below:

Drug A:

After ½hr was 32±5 sec

After 1 hr was 35±6 sec

After 2 hr was 34±4 sec

Drug B:

After ½hr was 31±6 sec

After 1 hr was 34±6sec

After 2 hr was 33±5sec

The drug C at the doses administered did not show any anti-histaminic effect. As seen from various experiments cited in example 5, two facts emerged distinctly. Firstly the liquid oral dosage forms of the herbal composition of the invention is more effective than a solid dosage form. Secondly, it is evident that sugar content in the formulation has no role in the degree of efficiency. Both the formulations containing 20%w/v and 1%w/v of residual sugars respectively are found equally well in the experimental studies. This specific observation shows that, the herbal composition can be prepared with low sugar content which benefits diabetic and calorie conscious persons having bronchial asthma and the other one for persons (non-diabetics) having bronchial asthma.

To assess the clinical efficacy of the proposed herbal composition in human subjects, a clinical study was also conducted.

### EXAMPLE 6

Clinical study

The design of study is double blind clinical study, in which Sample A is the drug while the Sample C is a placebo with similar appeal and taste.

Patients having the history of bronchial asthma of 0-5 years duration of either sex, aged between 20-50years, 15 in each group (A&B, total 30 patients) were selected randomly. Hereditary factors, occupation, season, Economic status, Food habits etc. were considered. The patients suffering from chronic lesions, cardiac problems, Tuberculosis, Pregnancy, Anaemia, Emphesema, Lung abscess, Corpulmonale, Bronchiectasis etc were excluded.

The patients were divided randomly into two groups A and B containing 15 in each. Group A patients were treated orally with Drug A at the dose of 10 ml thrice daily half an hour after food. Similarly, Group B patients were assigned to therapy with drug B, continuously for 60 days.

The following subjective and objective parameters were employed the clinical efficacy of the herbal composition.

Subjective parameters

a) Cough, Running nose, Wheezing, Breathlessness

b) Appetite

C) Capacity to perform routine duties

d) Felling of well being

Objective parameters

A) Spirometry

I) Forced Expiratory Volume (FEV)

II) Peak Expiratory Flow Rate (PEFR)

III) Erythrocyte Sedimentation Rate (ESR)

B) Haematology

IgE

Observations carried during clinical study

a) Cough, Running nose, Wheezing, Breathlessness

b) Appetite

c) Capacity to perform routine duties

d) Felling of well being

Follow up was done by-weekly to note down the clinical changes for the period of 60 days

Overall relief rate in Subjective Parameters with Drug A

| | Relief Rate | | |
|---|---|---|---|
| | (% age of patients) | | |
| Signs/Symptoms | Excellent | Moderate | Poor |
| Cough, Running nose, Breathlessness & | 80% | 18% | 02% |
| Wheezing | | | |
| Appetite | 90% | 10% | — |
| Capacity to perform routine duties | 96% | 04% | — |
| Feeling of well being | 88% | 10% | 02% |

Overall relief rate in Subjective Parameters with Drug B

| | Relief Rate | | |
|---|---|---|---|
| | (% age of patients) | | |
| Sign/Symptoms | Excellent | Moderate | Poor |
| Cough, Running nose, Breathlessness & | — | 4% | 96% |
| Wheezing | | | |
| Appetite | — | 6% | 94% |
| Capacity to perform routine duties | — | 07% | 93% |
| Feeling of well being | — | 10% | 90% |

Objective Parameters

Besides subjective parameters, the patients were also evaluated on the basis of objective parameters at different time intervals during the therapy with the formulation. They are summarized below:

1. Forced Expiratory Volume (FEV): FEV is a simple test to assess the following functional status of lungs. This value came down during an asthmatic condition. This value was assessed before the onset of treatment, middle and end of the treatment. There was a statistically significant improvement at both the points as compared to basic value of FEV. This observation was not seen in the patients receiving placebo sample.

2. Peak Expiratory Flow Rate (FEFR): FEV is a simple test to assess the following functional status of lungs. In the drug treated group there has been a consistent but marginal rise was observed. In the case of placebo treated group marginal reduction was observed at the end of the study.

3. IgE: The Immunoglobulin E (IgE) levels tend to increase in case of any allergy. Therefore it is used as a marker to measure the clinically improvement of a patient allergic to a subject/expression. It was seen that the mean value of IgE came down almost by 50% as compared to the mean value at base line level. In the case of placebo treated group there was a marginal increase in this value at the end of the therapy.

4. Erythrocyte Sedimentation Rate (ESR): ESR signifies any kind of inflammatory process taking place in the human body. This parameter was monitored before, middle and end of the treatment. It was seen in the drug treated group that there was a consistent reduction in the ESR value in each patient. On the other, the value remains more or less the same in the placebo treated group.

It should be noted that the above observations are as result of two months treatment, wherein the ventillatory capacity was seen to be increasing, while the tendency to allergy was noted to decrease over a period of time. These observations substantiate the subjective response noted by the patients tabulated above.

We claim

1. A oral liquid herbal composition useful in the management of asthma in diabetic and calorie conscious persons, said composition comprising a therapeutically effective amount of plant extracts selected from Solanum xanthocarpum, Albizzia lebbeck, Tribulus terrestris, Glycyrrhiza glabra, Pistachia integerrima, Adathoda vasica and Woodfordia fruticosa, and having self-generated ethanol to the extent of 7 to 11% v/v and not more than 1 to 3% w/w of sugar content.

2. A oral liquid herbal composition useful in the management of asthma in diabetic and calorie conscious persons, comprising extracts of plants selected from:

| | | |
|---|---|---|
| a. | Solanum xanthocarpum | 3-12% w/v, |
| b. | Albizzia lebbeck | 5-15% w/v, |
| c. | Tribulus terrestris | 3-12% w/v, |
| d. | Glycyrrhiza glabra | 1-10% w/v, |
| e. | Pistacia integerrima | 1-5% w/v, |
| f. | Adathoda vasica | 1-5% w/v, and. |
| g. | Woodfordia fruticosa | 1-7% w/v |

and having sugar content to the extent of 1 to 3% w/w and self-generated ethanol to the extent of 7 to 11% v/v, and, optionally, comprising extracts/powder of Elettaria cardamum 0.1 to 0.3% w/v, Piper longum 0.1 to 0.3% w/v, Mesua ferrea 0.1 to 0.3% w/v and Syzigium aromaticum 0.1 to 0.3% w/v.

3. A oral liquid herbal composition as claimed in claim 1, said composition comprising extracts of:

| | | |
|---|---|---|
| a. | Solanum xanthocarpum | 7% w/v, |
| b. | Albizzia lebbeck | 9% w/v, |
| c. | Tribulus terrestris | 7% w/v, |
| d. | Glycyrrhiza glabra | 5% w/v, |
| e. | Pistachia integerrima | 2% w/v, |
| f. | Adathoda vasica | 2% w/v, and. |
| g. | Woodfordia fruticosa | 3% w/v |

and having sugar content to the extent of 1 to 3% w/w and alcohol to the extent of 7 to 11% v/v, and optionally, comprising extracts/powder of Piper longum (0.075% w/v), Elettaria cardamomum (0.125% w/v), Syzigium aromaticu (0.075% w/v) and Mesua 0.100% w/v).

4. A composition as claimed in claims 1 or 2 wherein the plant parts used for preparation of the said composition are selected from fruits, leaves, stem and flowers of the plants.

5. A process for the manufacture of a oral liquid herbal composition, useful in the management of asthma, having self generated ethanol to the extent of 7 to 11% v/v and not more than 1 to 3% w/w of sugar content, said process comprising the steps of:
a. obtaining the extract of plant parts,
b. adding nutrients to the extract of step (a) in a manner such that the sugar content in the culture medium does not exceed 20% w/w,
c. adding micro-organisms capable of fermentation to the culture medium of step (b) and allowing it to ferment until the self-generated ethanol content thereof reaches 7 to 11% v/v, and
d. obtaining the herbal composition having total sugar content of not more than 3% w/w.

6. A process as claimed in claim 5 wherein the plants are selected from Solanum xanthocarpum, Albizzia lebbeck, Tribulus terrestris, Glycyrrhiza glabra, Pistachia integerrima, Adathoda vasica and Woodfordia fruticosa.

7. A process as claimed in claim 5 wherein the potentiating plants are optionally selected from Piper longum, Elettaria cardamomum, Syzigium aromaticu and Mesua.

8. A process as claimed in claim 5 wherein the plant parts are obtained by cold infusion or hot decoction methods.

9. A composition as claimed in claim 8 wherein the cold infusion method comprises the step of extracting the plant parts in water at a temperature ranging between 20 C. to 30° C.

10. A process as claimed in claim 8 wherein the hot decoction method comprises the step of extracting the plant parts in water by heating at a temperature in the range of 60 to 90° C.

11. A process as claimed in claim 5 wherein the nutrient in step (b) is a complex nutrient like jaggery or simple sugar like glucose, fructose or any other hexose sugar.

12. A process as claimed in claim 11 wherein the nutrient is in physical forms such as solid or liquid.

13. A process as claimed in claim 11 wherein the nutrients are added by fed batch or batch fermentation method.

14. A process as claimed in claim 11 wherein the nutrients are added in small amounts in regular intervals (at gradient quantities) in fed batch fermentation method.

15. A process as claimed in claim 14 wherein the addition of nutrients in fed batch fermentation method is such that each batch of nutrients added (each gradient) does not impart more than 5% v/v of sugar and the overall quantum of sugar added in the entire process does not exceed 20% w/w.

16. A process as claimed in claim 11 wherein the nutrients are added to the medium in the beginning at once in batch fermentation method.

17. A process as claimed in claim 5 wherein the microorganisms for fermentation comprise micro-organisms obtained from conventional sources like Woodfordia fruticosa, pure cultures such as baker's yeast, alcohol producing strains of Saccharomyces sp or strains of Saccharomyces cereviceae such as DRF-UDS-004/Wf, DRF-UDS-016/Wf, DRF-UDS-017/Wf or a combination thereof.

18. A process as claimed in claim 5 wherein in step (c) the culture medium is incubated at a temperature ranging between 20° C. to 37° C. for 2 to 40 days in anaerobic conditions maintaining the pH of the medium from 4 to 6.

19. A process as claimed in claim 5 wherein the temperature is maintained at 30° C.

20. A process as claimed in claim 5 wherein the incubation is effected preferably for 4 days.

21. A process as claimed in claim 5 wherein the pH of the culture medium is maintained at 4.5.

22. A process as claimed in claim 5 wherein the final sugar content in the herbal composition is not more than 1% w/w.
